(19) United States
(12) Patent Application Publication
(10) Pub. No.: US 2010/0015219 A1
(43) Pub. Date: Jan. 21, 2010

(54) PHARMACEUTICAL COMPOSITIONS OF ACTIVE SUBSTANCES DIFFICULT TO IMPROPERLY DIVERT FROM THEIR INTENDED ROUTE OF ADMINISTRATION

(75) Inventors: Marie-Pierre Berleur, Neuilly Sur Seine (FR); Jean-Claude Chaumeil, Saint Michel Sur Orge (FR); Francois Guyon, Paris (FR); Jean-Jacques Houri, Le Perreux Sur Marne (FR); Thierry Kin, Paris (FR); Gilles Le Pallec, Ste Genevieve Des Bois (FR); Elisabeth Moreau, Paris (FR)

Correspondence Address: YOUNG & THOMPSON, 209 Madison Street, Suite 500, Alexandria, VA, 22314, US

(73) Assignees: BOUCHARA-RECORDATI, LEVALLOIS-PERRET (FR); ASSISTANCE PUBLIQUE HOPITAUX DE PARIS, PARIS (FR)

(21) Appl. No.: 12/375,452

(22) PCT Filed: Jul. 25, 2007
(86) PCT No.: PCT/FR07/51724

§ 371 Date: Jan. 28, 2009

(30) Foreign Application Priority Data
Jul. 28, 2006 (FR) 0606956

(51) Int. Cl. A61K 9/48; A61K 31/4355; A61K 31/44; A61K 31/135; A61K 31/22; A61P 25/36

(52) U.S. Cl. 424/451; 514/279; 514/282; 514/648; 514/546

(57) ABSTRACT

A solid pharmaceutical composition in the form of a gel capsule containing at least one active ingredient and also at least one gelling agent chosen from cellulose derivatives such as hydroxyethylcellulose, hydroxypropylcellulose, hydroxypropylmethylcellulose, methylcellulose, ethylmethylcellulose, sodium carboxymethylcellulose (CMC) and calcium carboxymethylcellulose, and at least one disintegrating agent, the gelling agent and the disintegrating agent being present in a ratio by weight of 0.3 to 0.7/1 and the content by weight of gelling agent(s) being from 10% to 20% of the composition, and method for preparing same.

The present invention relates to pharmaceutical compositions of active substances, in particular categorized as narcotics, that are difficult to improperly divert from their intended route of administration.

Many narcotic substances are used in human medicine, and in particular opiates such as levacetylmethadol, buprenorphine, hydromorphone or methadone, which can be used in opiate substitution treatment or in the treatment of pain.

The satisfactory bioavailability of most of these substances when taken orally has led to the development of various pharmaceutical forms suitable for this route of administration.

The narcotic nature of these substances can lead individuals who are drug users to improperly divert these medicaments from their normal route of administration, in particular in order to use them by parenteral injection and, in particular, intravenously. For this, it may be sufficient to dissolve the solid form in a small volume of water.

Other, non-narcotic, substances could be improperly diverted from their normal route of administration. These are, for example, psychotropic active substances. The easiest for drug users to use are gel capsules since tablets have the drawback of having to be crushed before dissolution.

It would be desirable to have solid pharmaceutical compositions in the form of gel capsules (also known as hard capsules) of active substances, in particular categorized as narcotics, that are difficult to improperly divert from their intended route of administration.

It would also be desirable for said solid pharmaceutical compositions to maintain good bioavailability.

WO 00/38649, for example, describes solid pharmaceutical compositions in the form of tablets, the composition of which, after the tablets have been introduced into a drink of 20 ml or more, produces either insoluble particles, unsuitable for injection, or floating of the tablets.

Now, after a great deal of research, the applicant has developed a solid pharmaceutical composition in the form of a gel capsule, in particular for oral administration, producing a gel in the presence of water, in particular a small amount of water, compatible with a parenteral injection, which is entirely satisfactory.

For this reason, a subject of the present application is a solid pharmaceutical composition in the form of a gel capsule, containing at least:
an active ingredient and also
at least one gelling agent chosen from cellulose derivatives such as hydroxyethylcellulose, hydroxypropylcellulose, hydroxypropylmethyl-cellulose, methylcellulose, ethylmethylcellulose, sodium carboxymethylcellulose (CMC) and calcium carboxymethylcellulose, and
at least one disintegrating agent,
   the gelling agent and the disintegrating agent being present in a ratio by weight of 0.3 to 0.7/1 and the content by weight of gelling agent(s) being from 10% to 20% of the composition.

The gelling agent and the disintegrating agent are present in a ratio by weight of one to the other of 0.3 to 0.7/1, advantageously of 0.35 to 0.65/1, preferably of 0.40 to 0.60/1, especially of 0.45 to 0.55/1, most particularly of approximately 0.5/1.

The solid pharmaceutical composition may be any known form of gel capsule, made of gelatin or other material used for the manufacture thereof, such as water-soluble cellulose ethers.

In the present application and in what follows, the term “gelling agent” denotes an agent which gels or increases the viscosity in aqueous solution.

The gelling agent is chosen from cellulose derivatives such as hydroxyethylcellulose, hydroxypropylcellulose, hydroxypropylmethylcellulose, methylcellulose, ethyl-methylcellulose, sodium carboxymethylcellulose (CMC) and calcium carboxymethylcellulose, especially calcium carboxymethylcellulose and sodium carboxymethyl-cellulose (CMC), in particular the latter. Alginic acid or salts thereof can also be used.

The content by weight of gelling agent(s) of from 10% to 20% will preferably be from 10% to 15%, especially from 10% to 12%, most particularly approximately 10% of the total composition. Those skilled in the art will be able to adjust said content according to the choice of the gelling agent. It will generally not exceed 20% by weight, preferably not exceed 15% by weight, especially not exceed 12% by weight, in particular not exceed 10% by weight of the total composition.

For example, several grades of CMC are commercially available. These CMCs differ from one another by virtue of their chemical and physical characteristics. A concentration of 4.0% to 6.0% of CMC is generally required to allow the formation of a gel in aqueous solution. The use of a CMC grade with a low degree of substitution at a concentration of 1% makes it possible, for example, to obtain a gel of high viscosity, 2500-4500 mPa·s, compatible with the desired objective.

In the present application and in what follows, the term “disintegrating agent” denotes a disintegrating or dispersing agent.

The disintegrating agent is, for example, sodium carboxymethylstarch, calcium carbonate, alginic acid or else certain silicate complexes (aluminum magnesium silicate), preferably calcium carbonate or sodium carboxymethylstarch, and in particular the latter.

The content by weight of disintegrating agent(s) will advantageously be from 20% to 40%, preferably from 20% to 30%, most particularly 20% of the total composition.

It should be noted that the invention advantageously uses proportions of disintegrating agent, for example sodium carboxymethylstarch (CMS), which are higher than the customary use (usual concentration of 2% to 8%). The disintegrating agent enables a more rapid dispersion of the mixture of powder and thus wetting of the powders, promoting gelling of the mixture.

Under preferential conditions for use of the invention, the content of gelling and disintegrating agent is included in the correct proportions to allow immediate gelling of the powder of a solid pharmaceutical composition above, after addition of an aqueous solution, preferably of water, i.e. in less than 1 minute, preferably in less than 30 seconds, particularly in less than 20 seconds, most particularly in less than 15 seconds after the beginning of agitation of the mixture of the powder and of the aqueous solution. The agitation is carried out by alternately turning a hemolysis tube end-over-end manually, said tube having a volume of 5 ml and containing a mixture of 150 mg of the powder with 3 ml of water, at a speed of fifty 180° end-over-end turns of the container per minute, at ambient temperature. The volume of 3 ml is in particular the approximate volume that drug addicts use to inject themselves with a narcotic provided in solid form.

The solid pharmaceutical composition of the invention may comprise, by way of active ingredient, pharmacologically active organic compounds, and preferably narcotic substances used in medicine, especially human medicine, and particularly opiates such as levacetylmethadol, buprenorphine, hydromorphone or methadone.

The narcotic substances are in particular the active substances that can be used in human therapy and are listed in Annexes I, II, III and IV of the Order of Feb. 22, 1990, determining the list of substances categorized as narcotics (O.J. of the French Republic of Jun. 7, 1990).

By way of active substances categorized as narcotics and listed in Annex I, mention may be made, for example, of methadone, hydromorphone and oxycodone, by way of active substances categorized as narcotics and listed in Annex II, mention may be made, for example, of codeine, dextropropoxyphene and dihydrocodeine, by way of active substances categorized as narcotics and listed in Annex III, mention may be made, for example, of gamma-hydroxybutyric acid, methaqualone and methyl-phenidate, and by way of active substances categorized as narcotics and listed in Annex IV, mention may be made of nabilone and its salts, and tetrahydrocannabinols and their salts.

The non-narcotic substances are, for example, the active substances categorized as psychotropic substances that can be used in human therapy and are listed mainly in Tables III (having a moderate to high therapeutic value according to the Vienna Convention) and IV (having a low to high therapeutic value according to the Vienna Convention) according to the Order of Feb. 22, 1990, determining in France the list of substances categorized as psychotropic substances.

By way of active substances categorized as psychotropic substances and listed mainly in Table III, mention may be made, for example, of buprenorphine and flunitrazepam, and by way of active substances categorized as psychotropic substances and listed mainly in Table IV, mention may be made of barbiturates such as, for example, allobarbital, barbital, phenobarbital, benzodiazepines such as, for example, diazepam, lorazepam or tetrazepam, benzodiazepine-related hypnotics such as, for example, zolpidem, and other anxiolytics such as, for example, meprobamate.

This may also involve, both for the narcotic substances and for the non-narcotic substances, their isomers in the cases where they may exist, in accordance with the corresponding chemical formula of said substances, the esters and ethers of said substances or of said isomers, the various salts of said substances, of their isomers, and of their esters and ethers.

In the composition of the invention, taking into account the customary dosages of the active ingredient used, the content by weight of active ingredient may vary within the range of advantageously from 0.3% to 54%, preferably from 0.5% to 40%, especially from 0.5% to 30.0%, most particularly from 0.6% to 27.0% of the total composition. By way of example, the amount of a unit dose of active ingredient may range from 1 mg to 40 mg for methadone hydrochloride.

In the composition of the invention, the active ingredient will also generally be mixed with at least one inert excipient, often predominant by weight, included as a diluent, such as, for example, calcium phosphate, lactose, mannitol, microcrystalline cellulose or starch.

The active substance would also generally be mixed with at least one lubricant for promoting flow of the powder during industrial manufacture, such as, for example, talc, corn starch, anhydrous colloidal silica or any other equivalent agent.

The active ingredient will also generally be mixed with at least one lubricant for reducing interparticle friction, such as stearic acid or salts thereof (calcium stearate, magnesium stearate), sodium lauryl sulphate or any other agent providing similar properties.

The content by weight of lubricant(s) will advantageously be from 0.25% to 5%, preferably from 0.5% to 2.5%, especially from 0.75% to 1.5%, most particularly from 0.8% to 1.2% of the total composition.

Any other additive compatible with the composition and the intrinsic properties of the pharmaceutical form, such as anti-agglomerating agents, antioxidants, dyes, vitamins, mineral salts, sapidity agents, smoothing agents, assembling agents or isolating agents, or mixtures thereof, can be envisioned in the development of the composition above.

The chosen excipients in the composition will advantageously be chemically inert with respect to the active substance so as to make it possible to obtain a pharmaceutical form which is stable over time, under normal storage conditions.

From the physical point of view, the generally predominant excipients (diluents, gelling agents and disintegrating agents) will preferably have a suitable particle-size profile.

For example, 90% of the particles will have a size of less than approximately 130 μm and 50% of the particles a size of greater than approximately 50 μm so as to allow a homogeneous mixture with the active ingredient, of which 90% of the particles are less than 380 μm with a distribution medium in the region of 180 μm (particle size evaluated by the laser diffraction technique).

The ratio by weight of the gelling agent to the disintegrating agent is an important aspect. The powdered mixture of various proportions of constituents was brought into contact with various volumes of water. The observations of the mixture of powders after having been brought into contact with water and agitated by vortexing for a period of 5 seconds were: before agitation, a dispersion of the mixture of powders or the appearance of agglomerates, after agitation, the formation of a gel or the solubilization of the mixture of powders, the maintenance of agglomerates or the persistence of powder. The specific ratio by weight of the gelling agent to the disintegrating agent makes it possible to obtain rapid disintegration of the powder mixture in the presence of water, generally without agitation, and the formation of a gel immediately after agitation.

A subject of the present invention is also a method for preparing a solid pharmaceutical composition as defined above, characterized in that the various constituents are mixed and are placed in a gel capsule so as to obtain the expected composition.

The gel capsules which are the subject of the present invention have very advantageous properties and qualities. While the extraction of active ingredients from any pharmaceutical form still remains possible, the solid pharmaceutical compositions above make it complex and lengthy and make it possible to prevent the practice of easy and rapid injection by means of the customary devices that drug users employ, after simply dissolving the content of the gel capsule in a small volume of water.

The specific ratio of the gelling agent to the disintegrating agent makes it possible to obtain rapid disintegration of the powder mixture in the presence of water, generally without agitation, and the formation of a gel immediately after agitation.

Bringing the powder derived from the composition into contact with an aqueous solution produces the formation of a gel which makes it impossible or at least very difficult to draw up the aqueous mixture. The criterion selected refers to standard EN ISO 7886-1 (“Sterile, non-reusable hypodermic syringes—Part 1: Syringe for manual use. Annex G: Method for testing the forces necessary to work the plunger—Table G1: Proposed values), the limits being between 0.25 and 1 daN.

The high viscosity of the solution obtained, in the presence of water, using the composition which is the subject of the patent, does not reduce the bioavailability of the active ingredient.

These properties are illustrated hereinafter in the experiment section. They justify the use of the compositions described above, in the manufacture of gel capsules intended for oral administration.

A subject of the present invention is also a method for making it difficult to inject a solid pharmaceutical composition comprising at least one active ingredient, characterized in that a mixture containing the active ingredient and also at least one gelling agent and at least one disintegrating agent is prepared, the gelling agent and the disintegrating agent being present in a ratio by weight of 0.3 to 0.7/1.

A subject of the present application is also the use of a mixture containing at least one gelling agent and at least one disintegrating agent, the gelling agent and the disintegrating agent being present in a ratio by weight of 0.3 to 0.7/1, for making it difficult to inject a solid pharmaceutical composition comprising at least one active ingredient.

The preferential conditions for use of the above solid pharmaceutical compositions described also apply to the other subjects of the invention covered above, in particular to the method for making it difficult to inject a solid pharmaceutical composition above and to the use of a mixture containing at least one gelling agent and at least one disintegrating agent, the gelling agent and the disintegrating agent being present in a ratio by weight of 0.3 to 0.7/1, for making it difficult to inject the content of a gel capsule comprising at least one active ingredient.

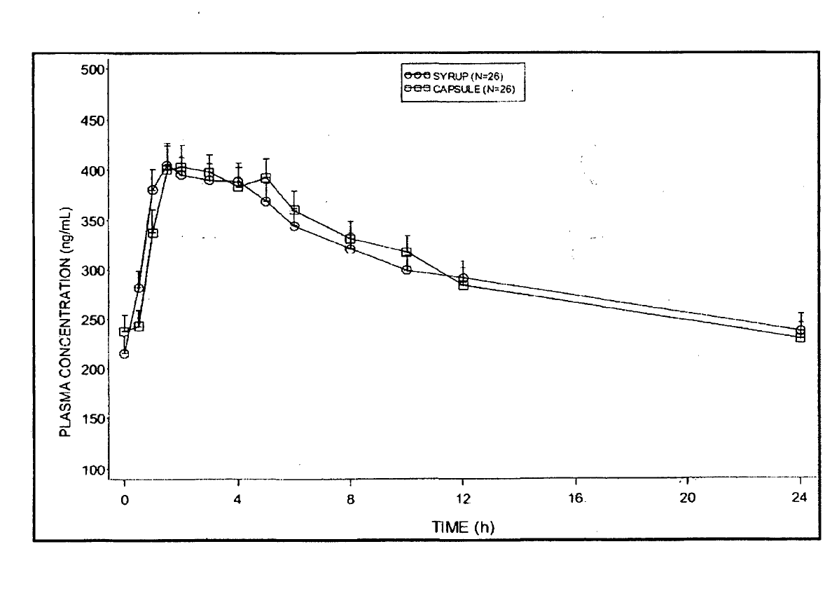

FIG. 1 represents the plasma profile of methadone after repeated administration in vivo, in humans, of 60 mg of methadone hydrochloride in the form of a syrup (reference) and in the form of a gel capsule (test formula) in 26 patients.

The examples which follow illustrate the present application. They were carried out with narcotic products for which the technique of the invention is of most interest, but other active ingredients could have been used.

### EXAMPLE 1

### Powder

A powder having the following composition by weight was prepared:

| | % |
|---|---|
| Methadone hydrochloride | 5.0 |
| Sodium carmellose | 10.0 |
| Sodium carboxymethylstarch | 20.0 |
| Magnesium stearate | 1.0 |
| Anhydrous colloidal silica | 1.0 |
| Lactose monohydrate | 63.0 |

### EXAMPLE 2

### Methadone Hydrochloride Gel Capsules

Gel capsules made of hard gelatin, containing a powder having the following composition by weight, were prepared:

| | % |
|---|---|
| Methadone hydrochloride | 20.0 |
| Sodium carmellose | 10.0 |
| Sodium carboxymethylstarch | 20.0 |
| Magnesium stearate | 1.0 |
| Anhydrous colloidal silica | 1.0 |
| Lactose monohydrate | 48.0 |

One gel capsule contains 100 mg of the above mixture.

The use of an amount of sodium carboxymethylcellulose greater than that of the present invention (>10%) leads to a decrease in the water content of the gelatin shell, making it brittle, thereby reducing its stability.

### EXAMPLE 3

### Test for Injection of Active Ingredients Extracted from a Composition of Example 2

The difficulty in injecting the medicament after it has been solubilized is based on tests of gel stability, of viscosity and of passage through syringe needles with the force applied to the plunger being measured.

The difficulty in injecting the formula developed was verified using a procedure adapted to the known practices of elicit drug users for solubilizing the content of gel capsules, then:
observing the difficulty in drawing up with a mounted or crimped needle of various interior diameters,
by measuring the concentrations of active substance of the solutions prepared starting from various dosages, with a view to calculating the extraction yields.

Only the solutions that pass through a needle were considered to be injectable.

The difficulty in drawing up the gel was studied using tuberculin syringes, mounted syringes and a syringe of large diameter (19 gauge) mounted on a 10 ml syringe, with the appearance of the gel being noted and the drawing-up time being measured.

Using the material normally employed by intravenous drug users (water, lemon, saliva, lighter, syringes, needles, spoons, devices of sterile kits distributed for this purpose and normally used by drug users), complete dissolution in the form of a fluid gel that passes through a needle of the largest interior diameter can be obtained only with a significant amount of water (more than 6 ml for 1 gel capsule) and particular conditions (breaking the gel after drawing up several times, exposing to heat, rapidly injecting). The fluid gel obtained according to this method passes through a needle of small interior diameter (tuberculin needle, insulin needle) but requires a syringe body of 10 ml and very gradual pushing of the plunger so as to avoid separation of the needle from the syringe body.

According to Standard EN ISO 7886-1 (“Sterile, non-reusable hypodermic syringes—Part 1: Syringe for manual use. Annex G: Method for testing the forces necessary for working the plunger—Table G1: Proposed values), the limits are between 0.25 and 1 daN. The values obtained for the content of the gel capsules brought into contact with water are much higher than these limits.

### EXAMPLE 4

### Bioavailability of the Active Ingredient Extracted from a Composition of Example 2

Tests were carried out under the standard study conditions recommended by the European Pharmacopeia.

The composition which is the subject of the patent allows the release of all the active ingredient in a medium representative of gastric fluid (0.1 mol/l hydrochloric acid) in a time of less than 30 minutes.

The high viscosity of the solution obtained, in the presence of water, using the composition which is the subject of the invention, does not reduce the bioavailability, as proved by comparing the curves of pharmacokinetics in volunteer individuals, obtained after repeated administration of 60 mg of methadone hydrochloride in the form:
of two gel capsules according to the invention (1 gel capsule of Example 2+1 gel capsule of Example 2 but containing 40 mg of methadone hydrochloride and accordingly less lactose),
of syrup,
   as illustrated in FIG. 1.

A solid pharmaceutical composition for oral administration prepared according to the invention cannot be injected by rapid solubilization (<2 min) in a small volume of water (1 ml) and yet does not reduce the bioavailability of the active ingredient.

The solutions obtained according to a long and complex optimized protocol have physical characteristics (opalescent fluid gel having a volume at least greater than 5 ml) that would require, in the case of injection of the preparation thus obtained, a material different from that proposed today by syringe exchange programs and dispensaries that drug users normally use, and, finally, a technical competence in the various steps of the manipulation, which despite everything results in a liquid preparation that is difficult to inject.

### EXAMPLE 5

### Gelling of Methadone Hydrochloride Tablets

The powder contained in the gel capsule of Example 2 is removed. It is placed at ambient temperature in a hemolysis tube having a volume of 5 ml, with 3 ml of water, and the mixture is agitated by turning the flask end-over-end manually at a speed of fifty 180° end-over-end turns of the flask per minute.

Gelling occurs in less than 30 seconds.

On the basis of the above examples and the teachings of the description, those skilled in the art can, without any particular burden of work, choose other gelling agents and other disintegrating agents or mixtures providing similar results.

1-10. (canceled)

11. A solid pharmaceutical composition in the form of a gel capsule containing at least one active ingredient and also at least one gelling agent chosen from cellulose derivatives such as hydroxyethylcellulose, hydroxypropylcellulose, hydroxypropylmethylcellulose, methylcellulose, ethylmethylcellulose, sodium carboxy-methylcellulose (CMC) and calcium carboxymethyl-cellulose, and at least one disintegrating agent, the gelling agent and the disintegrating agent being present in a ratio by weight of 0.3 to 0.7/1 and the content by weight of gelling agent(s) being from 10% to 20% of the composition.

12. The composition of claim 11, wherein the gelling agent and the disintegrating agent are present in a ratio by weight of one to the other of 0.40 to 0.60/1.

13. The composition of claim 11, wherein the gelling agent is chosen from sodium carboxymethylcellulose (CMC) and calcium carboxy-methylcellulose.

14. The composition of claim 11, wherein at least one disintegrating agent is sodium carboxymethylstarch.

15. The composition of claim 11, wherein the content by weight of gelling agent(s) is less than 15% by weight of the total composition.

16. The composition of claim 11, wherein the content by weight of disintegrating agent(s) is from 20% to 30% of the total composition.

17. The composition of claim 11, wherein the active ingredient is a narcotic substance used in medicine, such as levacetylmethadol, buprenorphine, hydromorphone or methadone.

18. The composition of claim 11, comprising at least one diluent and wherein 90% of the particles of the diluents, gelling agents and disintegrating agents have a size of less than approximately 130 μm and 50% of the particles a size of greater than approximately 50 μm, and 90% of the particles of the active ingredient have a size of less than 380 μm with a distribution medium in the region of 180 μm.

19. The composition of claim 11, wherein the content of gelling agent and of disintegrating agent is included in the correct proportions to allow gelling of the powder of the solid pharmaceutical composition, after addition of an aqueous solution, in less than 1 minute after the beginning of agitation of the mixture of the powder and of the aqueous solution.

20. A method for preparing a solid pharmaceutical composition in the form of a gel capsule containing at least one active ingredient and also at least one gelling agent chosen from cellulose derivatives such as hydroxyethylcellulose, hydroxypropylcellulose, hydroxypropylmethylcellulose, methylcellulose, ethylmethylcellulose, sodium carboxy-methylcellulose (CMC) and calcium carboxymethyl-cellulose, and at least one disintegrating agent, the gelling agent and the disintegrating agent being present in a ratio by weight of 0.3 to 0.7/1 and the content by weight of gelling agent(s) being from 10% to 20% of the composition, wherein the various constituents are mixed and are placed in a gel capsule so as to obtain the expected composition.

21. The method of claim 20, wherein the gelling agent is chosen from sodium carboxymethylcellulose (CMC) and calcium carboxy-methylcellulose.

22. The method of claim 20, wherein the gelling agent is chosen from sodium carboxymethylcellulose (CMC) and calcium carboxy-methylcellulose.

23. The method of claim 20, wherein at least one disintegrating agent is sodium carboxymethylstarch.

24. The method of claim 20, wherein the content by weight of gelling agent(s) is less than 15% by weight of the total composition.

25. The method of claim 20, wherein the content by weight of disintegrating agent(s) is from 20% to 30% of the total composition.

26. The method of claim 20, wherein the active ingredient is a narcotic substance used in medicine, such as levacetylmethadol, buprenorphine, hydromorphone or methadone.
